(19) United States
(12) Patent Application Publication
(10) Pub. No.: US 2009/0005858 A1
(43) Pub. Date: Jan. 1, 2009

(54) INTEGRATION OF MARKERS INTO BAR ARMS USING A WARM-FORMING PROCESS

(76) Inventors: Eugene Young, Union City, CA (US); Sanjay Shrivastava, Mountain View, CA (US); Russ Borg, Campbell, CA (US); Daniel H. Shumer, San Jose, CA (US); Mark Weiss, Redwood City, CA (US); Philip Simeon, San Jose, CA (US); Karim Osman, Mountain View, CA (US)

Correspondence Address: BAKER BOTTS L.L.P., 30 ROCKEFELLER PLAZA, 44th Floor, NEW YORK, NY, 10112-4498, US

(21) Appl. No.: 12/103,440

(22) Filed: Apr. 15, 2008

Related U.S. Application Data
(63) Continuation-in-part of application No. 11/768,477, filed on Jun. 26, 2007.

(51) Int. Cl. A61F 2/06

(52) U.S. Cl. 623/1.34

(57) ABSTRACT

A stent having a body capable of visualization under X-ray fluoroscopic techniques. The stent body includes radiopaque strut members. The stent body can also have a radiopacity gradient along the length of the stent body.

### CROSS REFERENCE TO RELATED APPLICATIONS

This application is a continuation-in-part application of U.S. patent application Ser. No. 11/768,477, filed Jun. 26, 2007, the content of which is incorporated herein by reference.

### BACKGROUND OF THE INVENTION

1. Field of Invention

The invention relates to a stent having a body with increased visualization capability. Particularly, the invention is directed to stent having radiopaque markers disposed on the strut members thereby providing a stent body that exhibits improved visualization under fluoroscopic equipment. More particularly, the radiopaque markers are formed on the strut members using a warm-forming process

2. Description of Related Art

Cardiovascular disease is prevalent in the United States and in other parts of the world. One manifestation of cardiovascular disease is atherosclerosis, which is the buildup of plaque (or fatty deposits) on the walls of blood vessels, such as coronary arteries. This buildup of plaque can grow large enough to reduce blood flow through the blood vessel. Serious damage results when an area of plaque ruptures and forms a clot, which travels to another part of the body. If the blood vessels that feed the heart are blocked, a heart attack results. If the blood vessels to the brain are blocked, a stroke results. Thus, atherosclerosis can be fatal for some people.

Typically, physicians treat atherosclerosis by implanting a tubular endoprosthesis such as a stent at the narrowed or blocked segment of the blood vessel, which widens and holds open the blood vessel. To perform this procedure the stent is delivered to the site of the lesion in the blood vessel by a catheter assembly, otherwise known as a stent delivery device. The stent delivery device enters the vasculature of the patient through the femoral artery and travels through a tortuous path to the site of the lesion. The physician positions the stent across the lesion and deploys the stent so that the stent forces the plaque against the inside wall of the blood vessel (or lumen) and maintains its expanded configuration so that the patency of the blood vessel is maintained.

In order to assist the physician in accurately positioning the stent across the lesion, conventional stents have been manufactured with markers coated or otherwise applied to the opposing ends of the stent. In this manner, the physician can visualize the ends of the stent during the procedure for accurate placement and deployment of the stent. Examples of such stents are disclosed in U.S. Pat. No. 6,464,721 to Boatman et al. and U.S. Pat. No. 6,022,374 to Imran et al., the disclosures of each of which is incorporated herein by reference thereto. The two ends of the stents as disclosed by Boatman and Imran allow for more accurate stent placement. One drawback, however, is that the conventional stents with markers do not enable visualization of the body of the stent. Additionally, any follow up angiographic procedures that need to be employed to visualize the stent in situ are improved. Other conventional stents include a marker extending from the proximal and distal ends of the stent body. Examples of such stents are disclosed in U.S. Pat. No. 6,503,271 to Duerig et al., the disclosure of which is incorporated herein by reference. Stents having radiopaque extensions such as disclosed in Duerig increase can interfere with the stent being crimped on a delivery device.

Thus, a stent body capable of visualization would be beneficial for a variety of reasons including easy assessment of the stent apposition to the vessel wall. Additionally, procedures that involve stent overlapping and stent side branch access can be performed with a greater level of confidence.

### SUMMARY OF THE INVENTION

The invention provides a stent having a body capable of visualization under fluoroscopic techniques. The stent includes an annular element comprising a set of interconnected strut members. Each strut member has a first end and opposing second end and a length therebetween. At least some of the strut members include a gap defined along its length and have radiopaque material disposed in the gap. The radiopaque material is disposed in the gap using a warm-forming process so that a radiopaque marker is formed.

The warm-forming process for forming the radiopaque marker includes the steps of heating the radiopaque material to a temperature below a recrystallization temperature of the material, and deforming the heated material plastically to fill the gap. The radiopaque material can be a metal such as tantalum.

The gap can extend through opposing surfaces of the strut member, thereby defining a complete opening therethrough. Alternatively, the gap be configured to extend partially through a surface of the strut member, if desired. In either embodiment, the radiopaque material is disposed using the warm-forming process such that the gap is entirely or partially filled with the radiopaque material. The radiopaque material is selected from the group including tantalum, platinum, iridium, gold, an alloy, or any combination thereof. The gap could also be filled with a polymer doped with any of the above materials or alloys. Further, the radiopaque material can be a solid material or alternatively comprises layers of material. The stent body can be formed of metal, metal alloy or polymeric material.

The gap defined in the strut member can have a predetermined shape along the length of the strut member. For example, the gap can be circular, linear, curvilinear, or polygonal. Some exemplary shapes include circles, rectangles, and/or diamonds.

The strut members can be configured to include a first width (W₁) and a second width (W₂) along the length of the strut member. The gap defined in the strut member can correspond to the first and second widths of the strut member, if desired. The strut member can alternatively include a plurality of widths along its length. In this manner, the strut member can include alternating first and second widths along its length. For example, the gap defined in the strut member can include a zig-zag configuration. Accordingly, the radiopaque material disposed in the defined shaped gap using the warm-forming process will have a complimentary configuration.

The stent body can include a variety of patterns, as would be known in the art. For example, the interconnected strut members can include a plurality of alternating strut members and crown members, thereby defining an undulating or serpentine pattern along the annular element. The interconnected strut members can be configured to have a “V” shape or a “U” shape depending on the existence and shape of the crown element.

When desired, the stent can be configured to include adjacent strut members and crowns configured to define a generally continuous wave pattern along a line segment parallel to a longitudinal axis of the stent body.

The stent has a tubular body which may consist of only a single annular element or instead a plurality of interconnected axially aligned annular elements. The axially aligned adjacent annular elements are connected at a plurality of connection sites. In one embodiment, a first set of alternating strut and crown members are axially aligned and axially offset from a second set of alternating strut and crown members. The first and second set of alternating strut and crown members can be in phase with each other or out of phase with each other, as desired.

The stent can include a proximal section, a distal section and an intermediate section therebetween. In this manner, at least one of the sections can include a greater amount of radiopaque markers than another section. Accordingly, for example but not limitation, the proximal section and the distal section of the stent body can have greater radiopacity than the intermediate section. Alternatively, the stent can be configured such that the intermediate section of the stent has greater radiopacity than the distal and proximal sections. In this regard, the stent can be configured to have a radiopacity gradient along a length thereof. For example, the radiopacity gradient can increase distally across the length of the stent. The increasing gradient can be a continuous gradient or a stepped gradient, if desired.

The advantages of radiopaque strut members in a stent include more precise placement of the stent under X-ray fluoroscopy, which allows the stent to be visualized not only during the procedure, but also post-procedurally. If the stent body can be visualized, the stent apposition to the vessel wall can be easily assessed. Additionally, procedures such as overlapping or side branching can be performed with greater confidence because the stent body can be visualized.

### BRIEF DESCRIPTION OF THE DRAWINGS

FIG. 1 is a schematic illustration of a prior art stent having a radiopaque marker tab extending from an end of the stent;

FIG. 2 is a schematic illustration of a prior art stent having radiopaque markers disposed in the crowns of opposing ends of the stent;

FIG. 3 is a schematic illustration of a segment of the stent having a gap defined in the strut member in accordance with an embodiment of the invention;

FIG. 4 is a schematic illustration of a segment of the stent having a radiopaque strut member in accordance with an embodiment of the invention;

FIGS. 5A and 5B are schematic illustrations of another embodiment of stent segment having a nestable radiopaque strut members in accordance with the invention;

FIG. 6 is a schematic illustration of another embodiment of a stent having radiopaque markers in accordance with the invention;

FIG. 7 is a schematic illustration of another embodiment of a stent in accordance with the invention;

FIG. 8 is a schematic illustration of another embodiment of a stent in accordance with the invention;

FIG. 9 is a schematic illustration of another embodiment of the stent in accordance with the invention;

FIG. 10 is a schematic illustration of an embodiment of the stent in accordance with the invention;

FIG. 11 is a schematic illustration of another embodiment of the stent body having a radiopacity gradient along its length; and

FIG. 12 is a flowchart of a warm-forming process for forming a radiopaque marker in a stent by filling a gap in the stent.

### DETAILED DESCRIPTION OF EMBODIMENTS OF THE INVENTION

While this invention may be embodied in many different forms, reference will now be made in detail to specific embodiments of the invention. This description is an exemplification of the principles of the invention and is not intended to limit the invention to the particular embodiments illustrated. For the purposes of this disclosure, like reference numbers in the figures shall refer to like features unless otherwise indicated.

It will be apparent to those skilled in the art that various modifications and variations can be made to the stent without departing from the spirit or scope of the invention. Thus, it is intended that the present invention include modifications and variations that are within the scope of the appended claims and their equivalents.

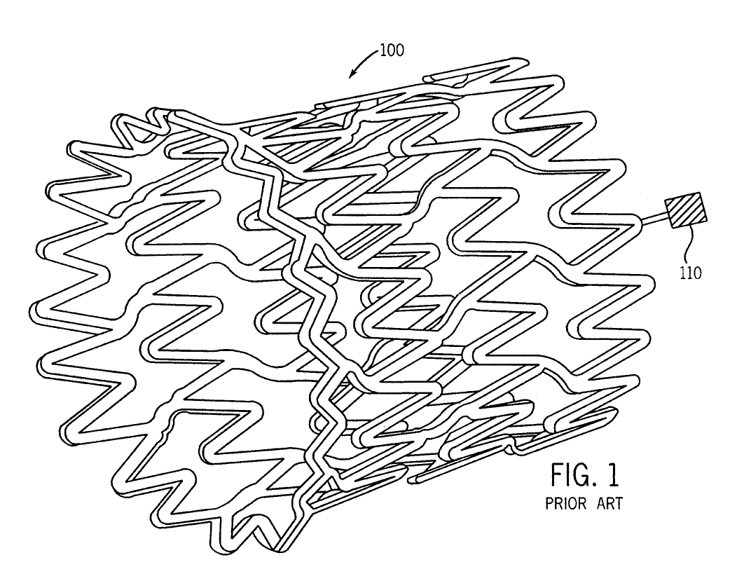

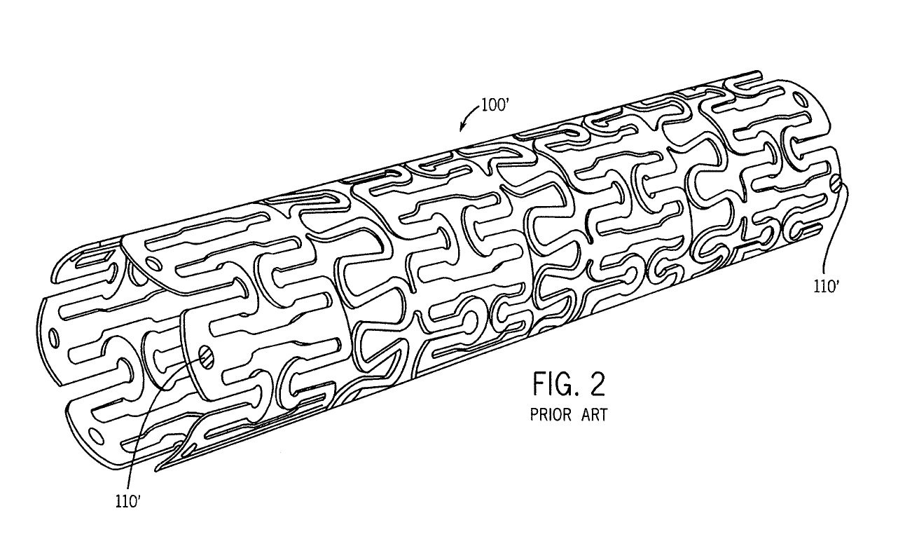

FIGS. 1 and 2 illustrate prior art stents having radiopaque material for increasing the radiopacity of the stent under X-ray fluoroscopy. As shown in FIG. 1, the prior art stent 100 includes a radiopaque tab δ 10 attached to the crown of a first annular element and extends laterally from the end of the stent body. Such added geometry to the stent body interferes with stent crimping. Further, although the end of the stent body includes visualization capability, the stent body cannot be viewed under X-ray fluoroscopy techniques.

An alternate prior art stent 100′, as shown in FIG. 2, includes a radiopaque marker 110′ disposed in the crown of the annular elements located at the distal and proximal ends of the stent body 100′. In contrast to the prior art stent 100, this stent 100′ maintains its longitudinal profile. However, only opposing ends of the stent body can be visualized under X-ray fluoroscopic techniques.

In accordance with the invention, a stent is provided having a body capable of visualization under X-ray fluoroscopy techniques. The stent is suitable for delivery within a body lumen of a mammal and can be configured for a variety of intralumenal applications, including coronary, peripheral, endovascular, biliary esophageal, urological, gastrointestinal applications.

Generally, the stent has a body, which includes a first set of interconnected strut members defining a first annular element. Each strut member includes a first end and a second end and has a length therebetween. At least some of the strut members include a gap defined along the length of the strut members. The gap is configured and shaped to receive radiopaque material, which is disposed in the defined gap using a warm-forming process that will be described herein later. Accordingly, the invention provides a radiopaque strut member to define a marker. In this manner, the entire stent body is capable of being visualized under fluoroscopic techniques.

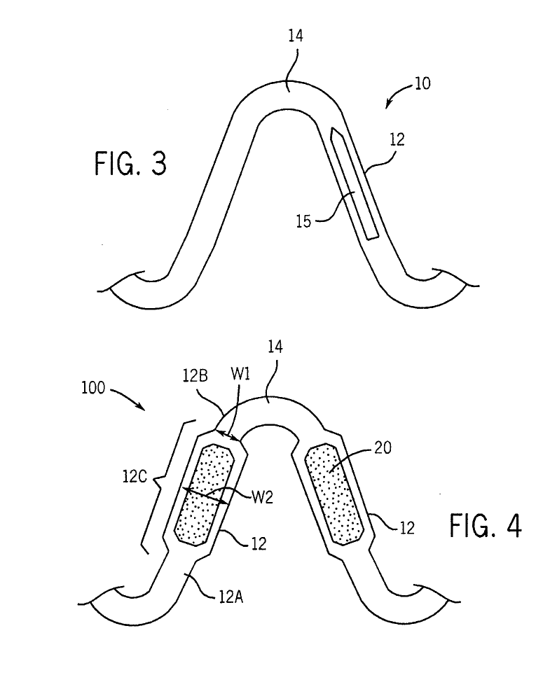

As schematically shown and depicted in FIG. 3, stent 10 has gap 15 defined in the strut member 12 by removing material from the strut member 12. The removal of material can be achieved by a variety of methods known in the art. For the purpose of illustration, the gap 15 can be defined by laser cutting or chemical etching techniques. The gap 15 can be configured such that it extends through opposing surfaces of the strut member to define an opening. In this manner, sufficient material is removed from the strut member to define the opening. Alternatively, the gap 15 can be configured to extend only partially through the strut member.

Radiopaque material 20 is integrated or formed in the gap 18 defined in the strut member 12. The radiopaque material 20 is integrated into the strut using a warm-forming process. Alternative methods of integrating the radiopaque material, however, can be utilized, as would be known in the art. A variety of radiopaque; materials can be used, such as tantalum, nitinol, platinum, iridium, gold, or alloys thereof. Additionally polymers doped with the above materials can be used. The selection of the radiopaque material is dependent on the material of the strut member. In this regard, the radiopaque material selected must have greater radiopacity than the material of the strut members of the stent body.

In accordance with an embodiment of the invention, as schematically depicted in FIG. 4, strut member 12 has a first end 12A, an opposing second end 12B, and a length 12C therebetween. Gap 15 is defined along the length 12C of the strut member and is configured and shaped to receive radiopaque material 20. Accordingly, the stent of the invention includes radiopaque strut members that define markers capable of visualization under X-ray fluoroscopic techniques.

Referring to FIG. 4 again, the strut members 12 can be configured to include a first width (W1) and a second width (W2) along its length 12C. As shown, gap 15 and radiopaque material 20 can be disposed using a warm-forming process along the length of the strut member 12 corresponding to the second width. Alternatively, the gap 15 can be disposed along the length corresponding to the first width or both widths, if desired.

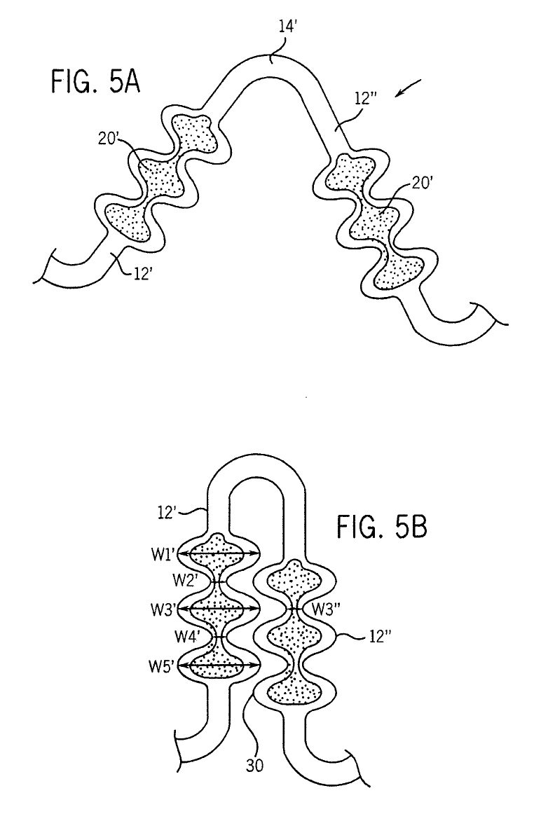

In another specific embodiment of the invention, the strut member is configured to include a plurality of widths along a length thereof. For example, the strut member 16 can include alternating first and second widths as schematically depicted in FIGS. 5A and 5B. In this manner, at least some of the plurality of widths can have the same width. Alternatively, if desired, each of the widths of the plurality can be different. In particular and as best seen in FIG. 5B, the width W3′ of one strut member 12′ can be shaped to compliment the width W3″ of an adjacent strut member 12″. In this manner, a first strut member 12′ can be configured be nested in a second strut member 12″ when the stent is in an unexpanded configuration.

The gap 15 defined in the strut member can have a predetermined shape. For example, the gap 15 can be linear, curvilinear, or circular. Alternatively, the gap 15 can be polygonal such as diamond-shaped, cube-shaped, or pyramidal shaped. The radiopaque material 20 disposed in the gap 15 is shaped using a warm-forming process to compliment or in some cases correspond to the defined gap.

The stent of the invention can be a self-expandable or balloon expandable stent having any configuration or pattern, as known to one skilled in the art. The stent body can comprise metal, metal alloy, or polymeric material. Some exemplary materials include Nitinol and stainless steel. Other complimentary materials include cobalt chromium alloy, ceramics and composites. Suitable polymeric materials include thermotropic liquid crystal polymers.

The stent body includes a first annular element including interconnected strut members. As embodied herein, the stent further includes a second set of interconnected strut members defining a second annular element.

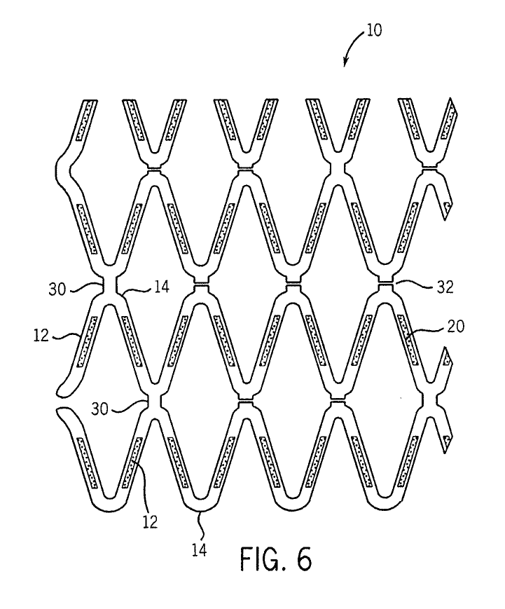

The interconnected strut members can be defined by alternating stent and crown members, which define the annular element. Each annular element generally defines a structure extending circumferentially along a longitudinal axis. First and second annular elements are axially aligned along a longitudinal axis and are associated with an adjacent annular element. For example as depicted in FIG. 6, the first and second annular rings can be associated with each other by a connection site 30 or alternatively by proximity 32.

A first annular element can include a first set of interconnected strut members that is out of phase with an axially aligned second set of interconnected strut members defining a second annular element. Alternatively, the axially aligned second set of interconnected strut members can be in phase with the first set of interconnected strut members, if desired. Further, the interconnected strut members of one annular element can be axially offset from the interconnected strut members of a second annular element.

In an embodiment, a plurality of connection sites define a connector column and the connected annular elements define a tubular structure. Each connection site is connected at one end to one annular element and at another end to an adjacent annular element. The number of connection sites can vary, e.g., decrease or increase, from connection column to adjacent connection column along the length of the stent body, as exemplified in U.S. Pat. Nos. 7,112,216 to Gregorich and 6,113,627 to Jang, the disclosures of which are incorporated herein by reference. Thus, the number of connection sites can continuously decrease or increase along a predetermined length of the stent body. Alternatively, the number of connection sites can be constant along a predetermined length of the stent body.

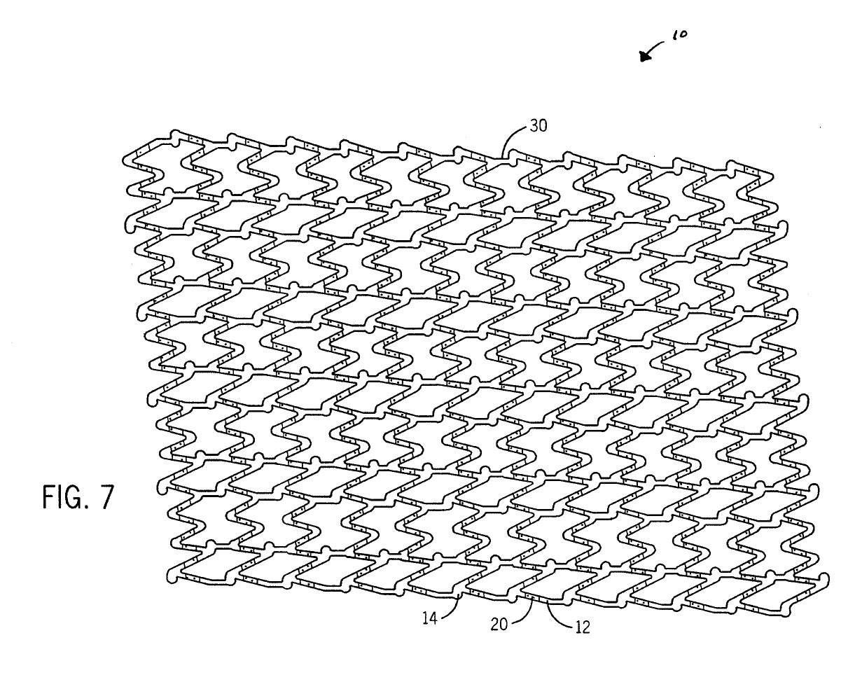

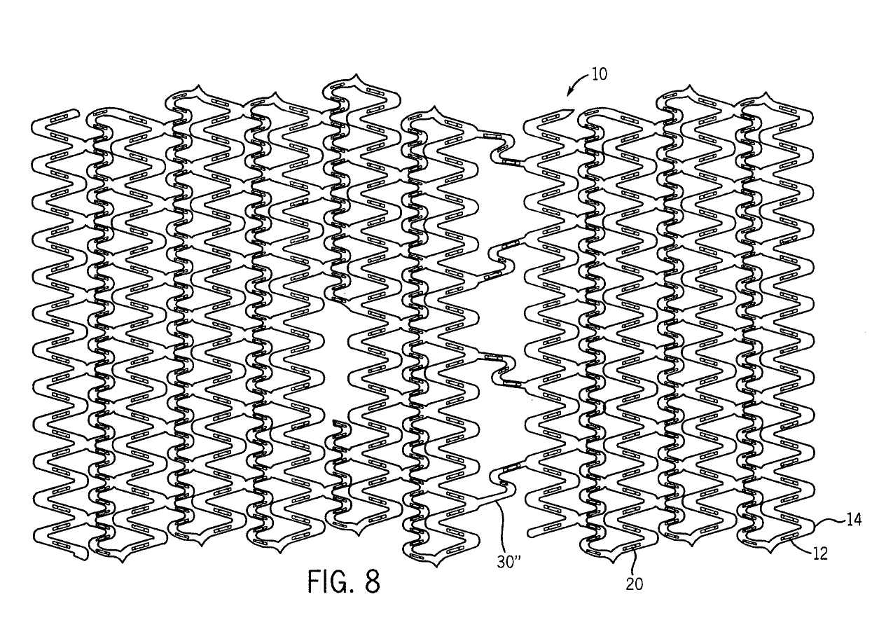

The connection sites can include a variety of configurations, lengths and widths. For example, the connection site can be a connection point 30, as depicted in FIG. 6. In other words, the crowns of adjacent annular rings can be joined together to form a connection site or point 30. Alternatively, the connection site can have a length to define a connector strut. The connector strut 30′ can have a substantially straight or linear configuration, as depicted in FIG. 7. Alternatively, the connector strut 30″ include at least one bend, i.e., non-linear, as depicted in FIG. 8.

In an embodiment of the invention, the stent body includes a first annular element comprising alternating strut and crown members axially aligned and out of phase with the alternating strut and crown members of a second annular element. The first and second annular elements are joined at a plurality of connection sites.

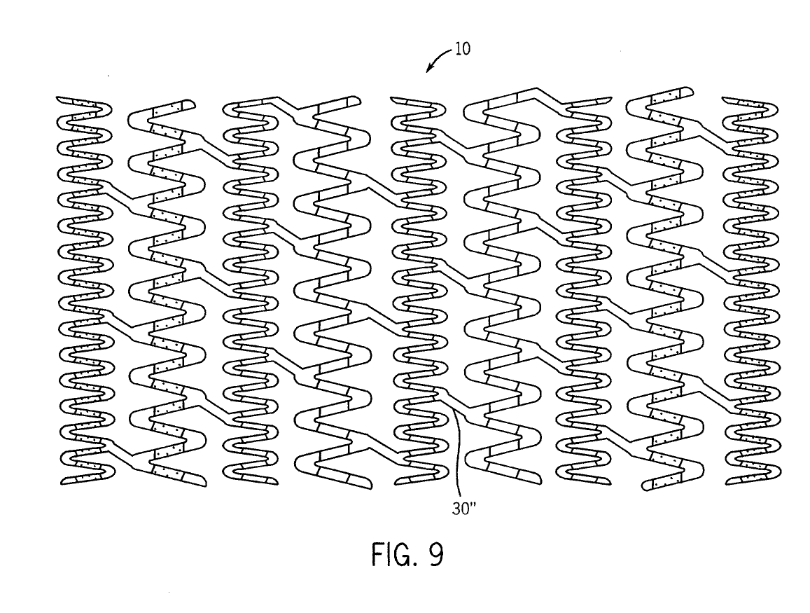

At least some of the connection sites extend from the center or from the side of the peak of one crown to the trough defined by the opposing crown. Alternatively, when the first set of alternating strut and crown members are in phase with the second set of alternating strut and crown members, the connection sites can extend from the peak defined by one crown to the peak defined by the opposing crown. The connection site 30′ can extend laterally from the first set of interconnected strut and crown members, as depicted in FIG. 7. Alternatively, the connection site 30″ can extend diagonally from the first set of interconnected strut and crown members to the second set of interconnected strut and crown members, as depicted in FIG. 9.

In another aspect of the invention, the length of the connection sites can vary along the length of the stent, as could the circumferential diameter of the connection sites. For example, the stent body can include shorter and wider connection sites in an intermediate section of the stent body compared to the proximal and distal sections of the stent body. In this manner, the stent has a greater outward radial force and compression resistance in the intermediate section of the stent body, as described in U.S. Pat. No. 7,060,091 to Killion, the entire content of which is incorporated herein by reference.

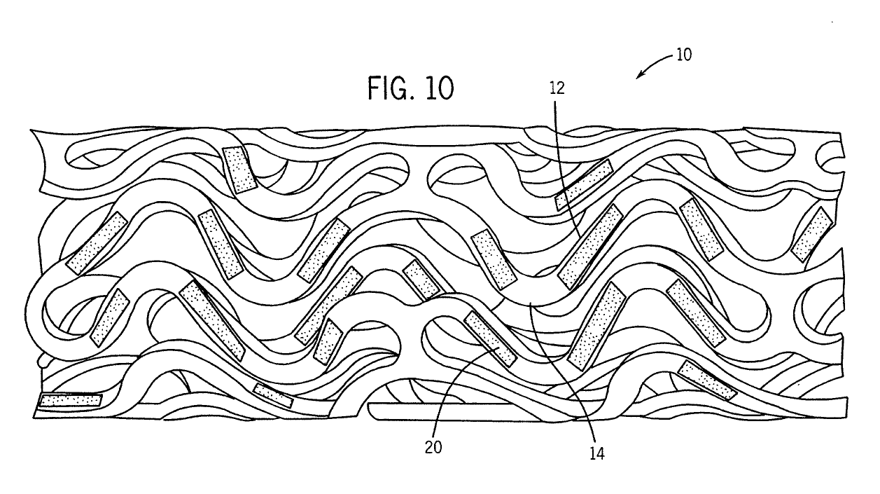

The alternating strut and crown members of the annular element can define an undulating configuration or pattern along a circumferential or a longitudinal path along the stent body. Adjacent annular elements of alternating strut members and crowns can define a generally continuous wave pattern along the longitudinal axis of the stent body, as depicted in FIG. 10.

In accordance with another aspect of the invention, the stent body includes a proximal section, a distal section, and an intermediate section therebetween. Each section includes an annular element having an interconnected set of strut members. At least some of the strut members include radiopaque material such that a radiopaque strut member is defined. The radiopaque material is integrated into the strut member using a warm-forming process.

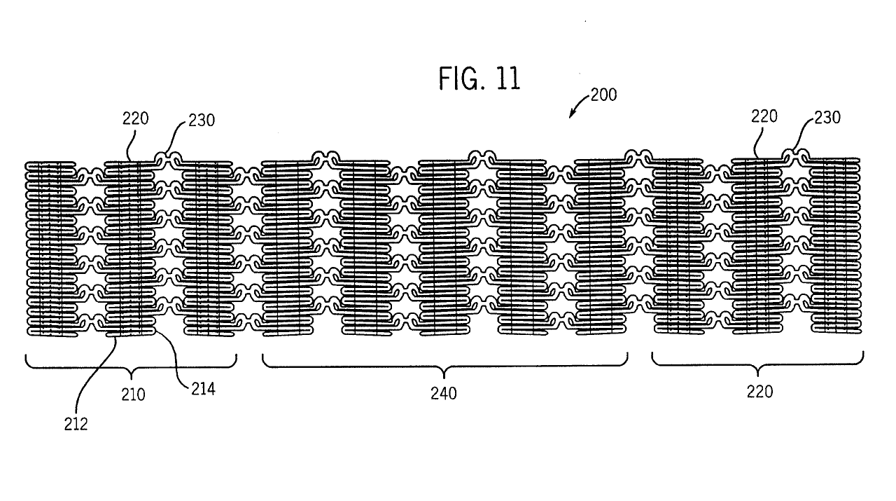

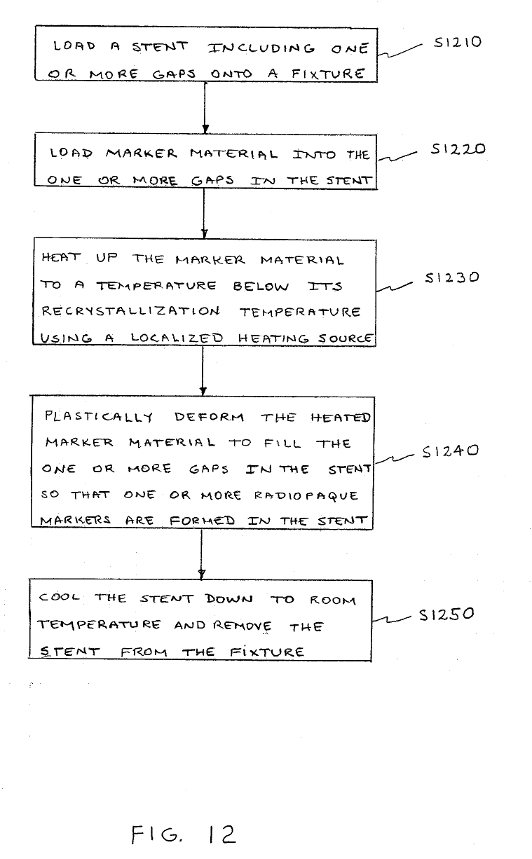

In one embodiment, as schematically shown and depicted in FIG. 11, at least one of the proximal 210, intermediate 230 or distal 220 sections of the stent body 200 has a greater radiopacity than the other sections. For example, the intermediate section 230 of the body can have greater radiopacity than the distal 220 and proximal 210 sections of the stent body 200 (not shown). Alternatively, the proximal 210 and distal sections 220 of the stent body 200 can have greater radiopacity than the intermediate section 230 of the stent body, as depicted in FIG. 11. In this regard, the proximal and distal sections can have the same degree of radiopacity.

The stent body 200 can be configured to include a radiopacity gradient across its length. For example, the radiopacity of the distal section 220 can be greater than both the intermediate 230 and the proximal 210 sections and further the radiopacity of the intermediate section 230 can be greater than the proximal section. In this manner, the radiopacity gradient can continuously increase distally across the length of the stent body 200. The radiopacity gradient can gradually, continuously increase in radiopacity across the stent body 200. If desired, however, the radiopacity gradient can be a stepped or abrupt increase of radiopacity across the stent body 200.

There are a couple of processes of forming metals that will be discussed below.

A hot-forming process, also referred to as a hot-working process, refers to a deformation process carried out under conditions and strain rates such that recovery processes occur substantially during the deformation process so that large strains are achieved with essentially no strain hardening. Examples of hot-forming processes are rolling, extrusion, or forging. Strain is usually large, e.g., 200% to 400%, and the temperature is usually greater than 0.6 Tm, where Tm is the melting temperature of the metal to be formed into a desired shape. Hot forming decreases the amount of energy required to deform the metal and its tendency to crack during deformation.

A warm-forming process, also referred to a warm-working process, refers to a process for deforming a metal plastically to form desired shapes using a temperature below the recrystallization temperature for a selected metal and above room temperature. Warm forming typically reduces the energy spent in the process while maintaining a good surface finish for the metal. Typical recrystallization temperature for a metal is about 0.3 Tm to 0.4 Tm.

By way of example, the melting temperature of tantalum, a radiopaque material, is 3,290 degrees K (3,017 degrees C.). A typical warm forming temperature for tantalum would be between 1,096 degrees K (823 degrees C.) and room temperature. A typical hot forming temperature for tantalum would be at or above 2,193 degrees K (1,920 degrees C.).

A warm-forming process is the preferred process for forming a radiopaque marker in a gap of a stent.

The warm-forming process for forming a radiopaque marker in a stent by filling a gap in the stent with a radiopaque metal, as discussed above, will now be described in detail with reference to FIG. 12. In step S1210 a stent including one or more gaps in loaded onto a fixture, and in step S1220 marker material is loaded into the one or more gaps in the stent. Then, in step S1230 the marker material is heated to a temperature below its recrystallization temperature using a localized heating source, and in step S1240 the heated marker material is plastically deformed to fill the one or more gaps in the stent so that one or more radiopaque markers are formed in the stent. Finally, in step S1250 the stent is cooled down to room temperature and removed from the fixture.

In addition to the specific embodiments claimed below, the invention is also directed to other embodiments having any other possible combination of the dependent features claimed below and those disclosed above. As such, the particular features presented in the dependent claims and disclosed above can be combined with each other in other manners within the scope of the invention such that the invention should be recognized as also specifically directed to other embodiments having any other possible combinations. Thus, the foregoing description of specific embodiments of the invention has been presented for purposes of illustration and description. It is not intended to be exhaustive or to limit the invention to those embodiments disclosed.

Many modifications, variations, or other equivalents to the specific embodiments described above will be apparent to those familiar with the art. It is intended that the scope of this invention be defined by the claims below and those modifications, variations and equivalents apparent to practitioners familiar with this art.

What is claimed is:

1. A stent comprising:
a body having a length and including a first annular element including a plurality of interconnected strut members, at least some of the plurality of strut members having a gap defined along a length thereof; and
radiopaque material disposed in the gap using a warm-forming process to define a marker.

2. The stent of claim 1, wherein the gap is formed by removing material from the strut member by laser cutting or chemical etching techniques.

3. The stent of claim 1, wherein strut members including opposing first and second surface, and further wherein the gap extends through the first and second surfaces to define an opening.

4. The stent of claim 3, wherein the radiopaque material completely fills the opening.

5. The stent of claim 1, wherein at least some strut members are configured to include a first width and a second width, the first width being different than the second width.

6. The stent of claim 5, wherein the gap has a first width and a second width corresponding to the first and second widths of the strut member.

7. The stent of claim 5, wherein the gap is defined in only one of the first or second widths of the strut member.

8. The stent of claim 5, wherein the strut member includes a plurality of widths to define a zig-zag configuration.

9. The stent of claim 8, wherein the gap has a corresponding zig-zag configuration.

10. The stent of claim 1, wherein the gap has a predetermined shape and the radiopaque material has a corresponding predetermined shape.

11. The stent of claim 10, the predetermined shape is selected from the group including rectangular, rounded, diamond-shaped, curved, and linear.

12. The stent of claim 1, wherein the interconnected strut members include alternating strut members and crowns.

13. The stent of claim 12, wherein the alternating strut members and crowns define an undulating pattern.

14. The stent of claim 1, wherein the first annular element is associated with a second annular element, the second annular element including a plurality of interconnected strut members.

15. The stent of claim 14, wherein the interconnected strut members of the first annular element are in phase with the interconnected strut members of the second annular element.

16. The stent of claim 15, wherein the interconnected strut members of the first annular element is axially offset from the interconnected strut members of the second annular element.

17. The stent of claim 1, including at least a first annular element comprising alternating strut and crown members associated with a second annular element comprising alternating strut and crown members, wherein adjacent strut members and crowns define a generally continuous wave along a line segment parallel to a longitudinal axis of the stent.

18. The stent of claim 1, wherein the first annular element and the second annular element are associated at a plurality of connection sites.

19. The stent of claim 18, wherein the stent includes a plurality of annular elements axially aligned and a plurality of connection sites that associate with adjacent annular elements, wherein the number of connection sites varies along a length of the stent.

20. The stent of claim 1, wherein the warm-forming process comprises the steps of heating the radiopaque material to a temperature below a recrystallization temperature of the material, and deforming the heated material plastically to fill the gap in the stent so that the marker is defined in the stent.

21. A stent comprising a stent body, the stent body including a proximal section, a distal section and an intermediate section therebetween, each section including at least one annular element of interconnected strut members, wherein at least some of the strut members include radiopaque material formed therein using a warm-forming process such that at least one section has greater radiopacity than the other sections.

22. The stent of claim 21, wherein the stent body includes a gradient of radiopacity along the length thereof.

23. The stent of claim 22, wherein the radiopacity gradient continuously increases distally along the length of the stent body.

24. The stent of claim 22, wherein the radiopacity gradient is a stepped increase over a length of the stent body.

25. The stent of claim 21, wherein each of the distal and proximal sections have greater radiopacity than the intermediate section.

26. The stent of claim 21, wherein the radiopaque material is disposed in a gap defined along a length of at least some strut members using the warm-forming process.

27. The stent of claim 26, wherein the warm-forming process comprises the steps of heating the radiopaque material to a temperature below a recrystallization temperature of the material, and deforming the heated material plastically to fill the gap in the stent so that a marker is defined in the stent.

28. The stent of claim 26, wherein the gap is formed by chemical etching or laser cutting techniques.

29. A warm-forming process for forming a radiopaque marker in a stent by filling a gap in the stent, comprising the steps of:
heating a radiopaque metal to a temperature below a recrystallization temperature of the metal; and
deforming the heated metal plastically to fill the gap in the stent so that the radiopaque marker is formed in the stent.

30. The warm-forming process of claim 30, wherein the radiopaque metal is tantalum.
